(19) United States
(12) Patent Application Publication
(10) Pub. No.: US 2026/0151320 A1
(43) Pub. Date: Jun. 4, 2026

(54) TOOTHPASTE TABLET COMPOSITION

(71) Applicant: HALEON UK IP LIMITED, Weybridge, Surrey (GB)

(72) Inventor: Simon KING, Weybridge, Surrey (GB)

(21) Appl. No.: 19/103,988

(22) PCT Filed: Aug. 7, 2023
(86) PCT No.: PCT/EP2023/071780

§ 371 Date: Feb. 14, 2025

(30) Foreign Application Priority Data
Aug. 25, 2022 (GB) 2212379.8

(51) Int. Cl. A61K 8/73; A61K 8/02; A61K 8/19; A61K 8/21; A61K 8/27; A61K 8/34; A61Q 11/00

(52) U.S. Cl. CPC A61K 8/731; A61K 8/0216; A61K 8/19; A61K 8/21; A61K 8/27; A61K 8/345; A61K 8/737; A61Q 11/00; A61K 2800/28

(57) ABSTRACT

The application discloses the composition of a superior toothpaste tablet with excellent organoleptic properties and suitable long-term stability.

### FIELD OF INVENTION

The invention relates to a superior composition for a toothpaste tablet. The tablet has excellent mouth feel and sensorial properties for a tablet, on par with a paste, and the toothpaste tablet has an excellent stability profile.

### BACKGROUND TO THE INVENTION

Toothpaste tablets have been on the market and known in the art for many years. Toothpaste tablets offer the convenience of consistent unit dosage and much more environmentally friendly packaging options than toothpastes, which are usually housed in difficult to recycle tubes.

Despite being known and available for years, toothpaste tablets have not really become a widely used product. This is largely due to the behaviour of the product in the mouth of the user. The compositions required for making good stable tablets have usually been accompanied by undesirable properties of poor mouth feel (often gritty and powdery) and poor foaming when compared with traditional tube-based toothpaste products.

It has been a desire to produce a toothpaste tablet that has the performance of a paste, so that more consumers can enjoy the tablet benefits, and more environmentally friendly packaging can be used.

### STATEMENTS OF INVENTION

In its broadest aspect the invention comprises a toothpaste tablet with superior organoleptic properties, comprising sorbitol, microcrystalline cellulose and either guar gum or cellulose gum or mixtures of the two thereof.

In a further embodiment the toothpaste tablet comprises; 50-80% by weight sorbitol powder; 10-30% by weight microcrystalline cellulose; and 0.1-10% by weight guar gum and/or cellulose gum.

In a further embodiment the tablet further comprises at least one abrasive.

In a further embodiment the at least one abrasive comprises between 1 and 10% by weight of the composition.

In a further embodiment the tablet further comprises at least one surfactant.

In a further embodiment the at least one surfactant comprises one or more surfactants from group comprising alkyl sulphates, betaines, taurates and mixtures thereof.

In a further embodiment the at least one surfactant comprises between 1 and 10% by weight of the tablet.

In a further embodiment the toothpaste tablet comprises: sorbitol powder 60-75% by weight, microcrystalline cellulose 15-25% by weight, guar gum and/or cellulose gum 0.1-3% by weight, at least one abrasive 2-8% by weight and at least one surfactant 1-5% by weight.

In a further embodiment the tablet comprises between 1-8% by weight of minor ingredients comprising; flavourings, colourings, preservatives, fragrances, pH modifiers, binders, glidants, further disintegrants and mixtures thereof.

In a further embodiment the toothpaste tablet further comprises a source of fluoride.

In a further embodiment the toothpaste tablet further comprises a zinc salt.

In a further embodiment the toothpaste tablet further comprises a bioglass.

In a further embodiment the toothpaste tablet further comprises a potassium salt.

In a further embodiment the toothpaste tablet further comprises an enzyme

In a further embodiment the toothpaste table further comprises a probiotic

In a further embodiment the toothpaste tablet further comprises a prebiotic.

In a further embodiment the toothpaste tablet further comprises a vitamin or mineral.

In a further embodiment the toothpaste tablet comprises a combination of two or more potentially incompatible actives.

In a further embodiment the toothpaste tablet comprises a combination of two or more of; a zinc salt, a potassium salt, a bioglass and stannous fluoride.

In a further embodiment the toothpaste tablet does not comprise guar gum.

In a further embodiment the toothpaste tablet does not comprise cellulose gum.

In a second aspect, the invention comprises the use of a toothpaste tablet of the first aspect of the invention in combination with a toothbrush for cleaning teeth.

In a further aspect the toothbrush is a manual or battery powered toothbrush.

In a third aspect, the invention comprises a method of making a toothpaste tablet of the first aspect of the invention; comprising the steps:
a. Adding the ingredients together in a solid form
b. Mixing the ingredients together until homogeneous
c. Compacting the mixture into tablets

In a further embodiment the compaction into tablets is carried out using 5 kN of compression force.

### DETAILED DESCRIPTION OF THE INVENTION

The applicants have devised a new formulation for a toothpaste tablet that overcomes the previous sensorial problems associated with currently commercially available toothpaste tablets. The toothpaste tablets of the present invention have a similar mouth feel and performance as a high-quality toothpaste.

The term “toothpaste tablet” as used herein, is meant to comprise a stable tablet product that is not intended for swallowing or ingesting. The toothpaste tablet is not intended for the administration of systemic therapeutic agents, but retained in the oral cavity for a sufficient time for it to breakdown in contact with saliva and make contact with substantially all of the dental surfaces and/or mucosal tissues.

The toothpaste tablet of the present invention is intended for use in combination with a toothbrush, manual or electric, for the purposes of cleaning teeth.

The core of the toothpaste tablets of the present invention is comprised of an optimised formula of just three main ingredients, sorbitol, microcrystalline cellulose and guar gum and/or cellulose gum.

These ingredients have been selected for their multifunctional benefits. Sorbitol is a bulking agent for the tablet but also provides a natural sweetness in the mouth. Guar gum and cellulose gum work as a binder for the tablets, but also provide superior mouth feel in use. Microcrystalline cellulose is a refined form of natural cellulose found in most plant materials. It is used in dehydrated form as both disintegrant and binder in pharmaceutical products.

The toothpaste tablets of the present invention are preferably non-aqueous. As used herein, the term “non-aqueous” means anhydrous or substantially free of water. The individual components of the non-aqueous composition may contain limited amounts of water as long as the overall composition remains substantially free of water.

The ingredients are preferably supplied in dry powder form and admixed thoroughly before the tableting process.

The amount of sorbitol in the tablets of the present invention is preferably between 40 and 85% by weight of the toothpaste tablet, preferably between 50 and 80% by weight, more preferably between 65 and 75% by weight.

A particularly preferred amount of sorbitol for the tablets of the present invention is around 70% by weight.

The sorbitol for the present invention could be sourced from a variety of different suppliers.

A particularly preferred sorbitol for the present invention is sorbitol powder is sold by Cargill under the C*PharmSorbidex P brand.

The amount of microcrystalline cellulose in the tablets of the present invention is preferably between 5% and 40% by weight, preferably between 10 and 30% by weight and most preferably between 15% and 25% by weight.

A particularly preferred amount of microcrystalline cellulose is about 20% by weight.

A particularly preferred microcrystalline cellulose for the tablets of the present invention is sold by JRS under the Vivapur® 102 brand.

Guar gum is an exo-polysaccharide composed of the sugar's galactose and mannose. Cellulose gum (or Carboxymethyl cellulose (CMC)) is a cellulose derivative with carboxymethyl groups (—CH₂—CO₂H) bound to some of the hydroxyl groups of the glucopyranose monomers that make up the cellulose backbone.

The toothpaste tablets of the invention utilise either guar gum or cellulose gum or mixtures of the two. The two gum types may be interchangeable in some instances.

A preferred embodiment comprises guar gum only.

A further preferred embodiment comprises cellulose gum only.

The amount of guar gum or cellulose gum (or mixtures of the two) for use in the toothpaste tablets of the present invention is preferably between 0.1% and 10% by weight, more preferably between 0.2% and 5% by weight, more preferably between 0.3% and 3% by weight and most preferably between 0.4% and 2% by weight.

A particularly preferred amount of guar gum or cellulose gum for the purposes of the present invention is about 0.5% by weight.

A particularly preferred source of guar gum for the purposes of the present invention is Avicel®CE-15. Sold by DuPont. This is a combination of 15% guar gum with 85% microcrystalline cellulose and is easy to handle.

A particularly preferred source of cellulose gum for the purposes of the present invention is Avicel® PC 611. This is a combination of 15% cellulose gum with 85% microcrystalline cellulose and is similarly easy to handle.

### Optional Ingredients

The three core ingredients provide the base chassis for the toothpaste tablets of the present invention, but they are capable of incorporating many other optional components.

The three core ingredients produce tablets with excellent properties of stability, mouthfeel in use and flexibility to allow inclusion of other useful oral health ingredients without effecting their overall performance.

The toothpaste tablets of the present invention may further comprise a wide range of further ingredients. For example, in some embodiments, the toothpaste tablets can optionally comprise a glidant, a disintegrant, an additional binder, an abrasive, a surfactant, a fluoride source, a flavouring, a whitening agent, a colourful, a preservative, a fragrance, a pH modifier, or any combination thereof.

Non-limiting examples of additional ingredients are described below.

The toothpaste tablets of the present invention may contain a glidant. A glidant is a substance that is added to a powder to improve its flowability. In tablet manufacture, glidants are usually added just prior to compression.

However, in some embodiments, the toothpaste tablets of the present invention do not need a glidant. Effective tableting performance, stability and in-use performance is achieved using just the three core ingredients.

In certain preferred embodiments, the tablets of the present invention may be glidant free.

Examples of glidants include ascorbyl palmitate, calcium palmitate, magnesium stearate, fumed silica (colloidal silicon dioxide), starch and talc.

Tablets often require the presence of a disintegrant for suitable performance in use. A disintegrant is an excipient that is incorporated into the table to promote their disintegration when they encounter a liquid or fluid.

In the case of toothpaste tablets the fluid or liquid will be a combination or saliva and water.

The tablets of the present invention comprise microcrystalline cellulose and do not require further disintegrants. However, an additional disintegrant may be added in some embodiments, if more rapid disintegration is desired.

A non-limiting list of possible additional disintegrants includes, starch, glycolates, alginates, sodium carboxy-methylcellulose (NaCMC) and carmellose sodium.

In some embodiments of the invention the toothpaste tablets may be free of all additional glidants and further disintegrants.

The toothpaste tablets of the inventions may further comprise an additional binder in some instances. Binders are excipients that hold the ingredients of a tablet formulation together, Binders ensure that tablets, powders, granules and others can be formed with the required mechanical strength. Moreover, they can give volume to low active dose tablets.

In some embodiments, the toothpaste tablets of the present invention do not require an additional binder. They are stable without the addition of a further dedicated binder. However, an additional binder can be incorporated if desired.

Non-limiting examples of binders suitable for toothpaste tablets include gelatin, glucose, lactose, cellulose derivatives-methyl cellulose, ethyl cellulose, hydroxy propylmethyl cellulose, hydroxy propyl cellulose and poly vinyl pyrrolidone (Povidone).

The tablets may comprise at least one abrasive to aid the cleaning of the tooth surface. Any abrasive suitable for use in oral care products may be included. The skilled person will be aware of many of these different compounds.

The invention is not limited to any particular abrasive.

Examples of suitable dental abrasives include silica abrasives such as those marketed under the following trade names Zeodent®, Sident®, Sorbosil® or Tixosil® by Evonik, Degussa, Ineos and Rhodia respectively. The silica abrasive, if used, should be present in an amount sufficient to ensure adequate cleaning of teeth by the dentifrice whilst not promoting abrasion of the tooth surface.

The abrasive may be a single component or a mixture of two or more abrasives. The use of different abrasives allows the skilled person to optimise the cleaning power of the tablets.

The total abrasive is generally present in an amount up to 10% by weight of the total tablet composition, for example from 1% to 10% by weight, and preferably at least 2% to 8%. For example, from 3% to 7% by weight and especially 4% to 6% by weight of the total composition. Ultimately the level of abrasive in the tablet is determined by the particular abrasive used and the level of abrasivity desired in the final product. The skilled person can adjust the levels as required.

Particularly preferred abrasives include hydrated silicas. And a particularly preferred grade is Zeodent® 124 by Evonik.

Another preferred abrasive is alumina. This may be used in whitening formulations. If used, alumina may be included between 0.2% and 2.5% by weight, preferably around 1% by weight of the toothpaste tablets.

The toothpaste tablets of the present invention may contain surfactants to provide additional foaming, cleaning and mouthfeel properties in some instances. The tablets of the present invention may contain at least one surfactant. The tablets of the invention may comprise a combination of two or more surfactants.

The skilled person is aware of the types of surfactants used in oral care compositions. The compositions are not limited to particular surfactants.

A suitable surfactant for use in the toothpaste tablets of according to the invention belongs to the class of compounds known as betaines. Structurally, betaine compounds contain an anionic functional group such as a carboxylate functional group and a cationic functional group such as quaternary nitrogen functional group separated by a methylene moiety. They include n-alkyl betaines such as cetyl betaine and behenyl betaine, and n-alkylamido betaines such as cocoamidopropyl betaine.

In one embodiment the betaine is cocoamidopropyl betaine, commercially available under the trade name Tego Betain®.

Suitably the betaine is present in an amount ranging from about 0.1% to about 10% by weight of the toothpaste tablet composition, for example from about 0.2% to about 3.0%, more preferably between about 0.3% to about 2.0% by weight of the toothpaste tablet composition.

Another surfactant for use in the toothpaste tablet compositions according to the invention is selected from a taurate based surfactant. Taurate surfactants useful in the present invention are salts of fatty acid amides of N-methyl taurine. They conform generally to the structural formula:

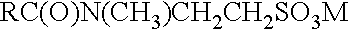

Where RC(O)— represents a fatty acid radical and M represents sodium, potassium, ammonium or triethanolamine. Fatty acids having carbon chain lengths of from 10 to 20, including those derived from coconut, palm and tall oil are used. In one embodiment the fatty acid is derived from coconut. In one embodiment, sodium salts are used.

In one embodiment the taurate is sodium methyl cocyl taurate. This taurate surfactant is sold under the trademark by Adinol CT® by Croda.

The taurate surfactant may be present in an amount from about 0.1% to about 10% of the toothpaste tablet composition. In one embodiment the taurate surfactant is present in an amount from about 0.1% to about 5% by weight of the toothpaste tablet composition. In one embodiment the taurate surfactant is present in an amount from about 0.5% to about 2.0% by weight of the toothpaste tablet composition.

Another class of surfactants suitable for the present invention are alkyl sulphate surfactants of the following structural formula:

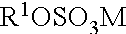

R¹ represents a fatty alcohol moiety and M represents sodium, potassium, ammonium or triethanolamine. Fatty alcohols having carbon chain lengths of from about 10 to about 20, including those derived from coconut, palm oil and tall oil. In one embodiment, the fatty alcohol is lauryl alcohol. In one embodiment, a sodium salt is used.

In one embodiment the alkyl sulphate is sodium lauryl sulphate (SLS).

The alkyl sulphate surfactant may be present in an amount from about 0.1% to about 10% of the non-aqueous composition. In one embodiment the alkyl sulphate surfactant may be present in an amount from about 0.1% to about 5% by weight of the toothpaste tablet composition.

In one embodiment the alkyl sulphate surfactant is present in an amount from about 0.5% to about 2.5% by weight of the toothpaste tablet composition.

The total amount of surfactant in the toothpaste tablets is preferably less than about 10%, more preferably less than 5% and most preferably less than 3% by weight of the tablet.

The tablets of the invention may have a source of fluoride ion, in some embodiments.

The fluoride source may be selected from sodium fluoride, stannous fluoride, sodium monofluorophosphate, potassium fluoride, ammonium fluoride, bis-(hydroxethyl) amino-propyl-N-hydroxyethyloctadecylamine-dihydrofluoride and mixtures thereof.

A particularly preferred source of fluoride ions for the present invention is sodium fluoride, stannous fluroride, sodium monofluorophosphate or mixtures thereof.

The dentifrices of the present invention may have any level of fluoride from 0.1 ppm to 10000 ppm.

The level of fluoride in oral care compositions in most jurisdictions is heavily controlled by state regulation. Levels of 450 ppm, 1150 ppm and 1426 ppm of fluoride are typical levels for commercial toothpastes. The present invention works well with these levels but is not limited to these amounts of fluoride.

The skilled person can readily determine the correct amount of fluoride source to add to get the correct levels.

The use of tablets for toothpaste allows for the controlled use of the correct amounts of actives for a single brushing.

In some embodiments, the toothpaste tablets of the present invention may contain many other minor ingredients.

The minor ingredients may be selected from the list of flavourings, whitening agents, colourings, preservatives, fragrances, pH modifiers and mixtures thereof.

The toothpaste tablets of the present invention may contain a total of 0.1%-10% by weight of minor ingredients.

The skilled person would be aware of examples of all of these ingredients that are suitable for oral care use.

The toothpaste tablets can be made in any size or shape required. A non-limiting example of a tablet of the present invention would be a flat round tablet, diameter of between about 0.8 cm and 1.8 cm, more preferably between 1.0 cm and 1.5 cm and most preferably between about 1.2 cm and 1.4 cm.

The weight of the toothpaste tablets can be adjusted to whatever the desired dosage requires. A non-limiting example of a suitable weight range for the toothpaste tablets of the present invention would be between 0.4 g and 2.0 g, more preferably between 0.6 g and 1.5 g and more preferably between 0.8 g and 1.2 g per tablet.

The toothpaste tablets of the invention may be of any shape desired. Preferably however the toothpaste tablets may be rounded for stability. The toothpaste tablets of the present invention may also be shaped to be stackable, such that they can be dispensed from the smallest amount of packing possible. For example, from a tube.

The toothpaste tablets of the present invention may be coated. The coating may be purely decorative or may be used to provide stability benefits.

### Toothpaste Tablet Preparation

The toothpaste tablets of the invention can be prepared by any standard tabletting technique.

The dry ingredients required for the final tablet, including all minor ingredients are added together, and mixed until homogeneous. The mixture is then added to the tableting press. The tableting press can be any press known to the skilled person for the pressing of tablets from dry powders.

The compressing force can also be adjusted to provide harder or softer tablets as the skilled person sees fit. The tablets of the present are particularly effective at a compression force of about 5 kN.

### Two or More Actives

There are many different actives that can be used in oral care products. Unfortunately, these are often incompatible in pastes. Either they react with each other, or are unstable in paste environments, be they aqueous or non-aqueous environments.

One of the advantages of the tablet format is that the product is completely solid and this allows for the combination of actives (potentially very mutually unstable actives) in a single product.

Non-limiting examples of desirable actives that can be awkward to co-formulate into a toothpaste include:
Zinc salts—frequently used in dental care products due to their anti-microbial and anti-inflammatory effects, positive effects on the freshness of breath and its ability to reduce or inhibit the formation of dental plaque and tartar. Zinc salts are ionic in character and can be difficult to stabilise in pastes, particularly in anhydrous formulations.
Potassium salts, in particular Potassium nitrate—frequently used in oral care compositions due to its desensitising properties. The compounds are also ionic and can be difficult to incorporate into pastes.
Stannous fluoride—frequently used in oral care products. Multiple benefits including fluoride delivery, plaque inhibition, gingivitis reduction and desensitisation. Stannous fluoride is very sensitive to moisture which makes aqueous formulations difficult.
Bioglass—Also used in oral care compositions for their desensitizing properties. In one embodiment the bioactive glass for use in the invention has a composition consisting of about 45% by weight silicon dioxide, about 24.5% by weight sodium oxide, about 6% by weight phosphorus oxide, and about 24.5% by weight calcium oxide. One such bioactive glass is available commercially under the trade name, NovaMin®, also known as 45S5 Bioglass®.

The tablets of the present invention are a highly stable chassis for all of the mentioned actives, alone or in combination.

In particular, the tablets of the present invention have been shown to be stable to:
Fluoride alone (single or multi source) including NaF and SnF₂
Potassium nitrate and SnF₂. (and NaF)
A bioglass and fluoride (in particular NaF)
And zinc salts and SnF₂

The invention is not limited to the above examples. The skilled person would be familiar with oral actives that have compatibility or stability difficulties in conventional pastes. The tablets of the invention are capable of stabilising a wide range of incompatible actives.

Other actives that may be included in the toothpaste tablets of the present invention are amino acids, vitamins, minerals, enzymes, probiotics and prebiotics. These may be included individually or in combination.

The amounts of additional actives included in the toothpaste tablets of the present invention by weight is not limited any particular levels. Typically, the amounts added will be determined by well-known efficacy levels for each active. The skilled person will be aware of the levels needed for efficacy.

Typically, the amounts of actives of included in the tablets will range from 0.1 to 15% by weight of the actives. The tablets of the present invention have been shown to be stable with a variety of actives at these levels.

### Results

The following compositions are all non-limiting examples of toothpaste tablets according to the invention.

### Example 1. General Core Formula of a Toothpaste Tablet of the Present Invention

| Ingredients | % w/w |
|---|---|
| Sorbitol Powder | 50-80 |
| Microcrystalline Cellulose | 10-30 |
| Guar Gum and/or Cellulose gum | 0.1-10. |

This is the core tablet composition of the present invention. This mixture provides tablets that have excellent stability and organoleptic properties. This tablet core can be augmented by the addition of additional orally suitable ingredients.

There is really no limit to the additional ingredients that can be added to the core formula to optimise the toothpaste tablets. All known components of toothpastes can be included.

The following further examples are not limiting to the scope of the invention. They are included to show how the general tablets of the present invention may be further optimised for different oral care purposes.

### Example 2—A More Preferred General Toothpaste Tablet Formulation of the Present Invention

| Ingredients | % w/w |
|---|---|
| Sorbitol Powder | 60-75 |
| Microcrystalline Cellulose | 15-25 |
| Guar Gum and/or Cellulose gum | 0.2-5 |
| At least one abrasive | 2-8 |
| At least one surfactant | 1-5 |

This formulation details a more preferred general tablet of the present invention that additionally comprises abrasive for cleaning performance and surfactant for foaming and mouth feel. The abrasive and the surfactant may be a single component or a mixture of two or more abrasives and two or more surfactants.

A preferred abrasive for example 2 would be a hydrated silica. And a preferred surfactant would be an alkyl sulphate based surfactant.

### Example 3—A Specific Guar Gum Example of a Tablet of the Present Invention

| Ingredients | % w/w |
|---|---|
| Sorbitol Powder (C*PharmSorbidex ® P) | 70.30 |
| Microcrystalline Cellulose (Vivapur ® 102) | 20.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate (SLS) | 2.00 |
| Sweetener - Stevia | 0.20 |
| Flavouring - Peppermint oil | 0.50 |

This specific tablet example incudes surfactant SLS and the abrasive is a hydrated silica.

### Example 4—an Anti-Caries Tablet

| Ingredients | % w/w |
|---|---|
| Sorbitol Powder (C*PharmSorbidex ® P) | 69.985 |
| Microcrystalline Cellulose (Vivapur ® 102) | 20.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate | 2.00 |
| Anticaries Agent - Sodium Fluoride | 0.315 |
| Sweetener - Stevia | 0.20 |
| Flavouring - Peppermint oil | 0.50 |

This composition details an anti-caries tablet of the present invention. This is equivalent to example 3, further including a fluoride source, in this case sodium fluoride.

### Example 5—a Stannous Fluoride Containing Tablet

| Ingredients | % w/w |
|---|---|
| Sorbitol Powder (C*PharmSorbidex ® P) | 67.846 |
| Microcrystalline Cellulose (Vivapur ® 102) | 20.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate | 2.00 |
| Chelating Agent - Sodium Tripolyphosphate | 2.00 |
| Antimicrobial Agent - Stannous Fluoride | 0.454 |
| Sweetener - Stevia | 0.20 |
| Flavouring - Peppermint oil | 0.50 |

Stannous fluoride is a well-known active that has a number of useful oral care properties as well as very well-known stability considerations.

Stannous fluoride is completely stable in the tablets of the present invention.

### Example 6—a Bioglass and Fluoride in Combination

| Ingredients | % w/w |
|---|---|
| Sorbitol Powder (C*PharmSorbidex ® P) | 64.985 |
| Microcrystalline Cellulose (Vivapur ® 102) | 20.00 |
| Desensitising Agent - Novamin ® 4516 | 5.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate | 2.00 |
| Anticaries Agent - Sodium Fluoride | 0.315 |
| Sweetener - Stevia | 0.20 |
| Flavouring - Peppermint oil | 0.50 |

### Example 7—Potassium Nitrate and Sodium Fluoride in Combination

| Ingredients | % w/w |
|---|---|
| Sorbitol Powder (C*PharmSorbidex ® P) | 64.985 |
| Microcrystalline Cellulose (Vivapur ® 102) | 20.00 |
| Desensitising Agent - Potassium Nitrate | 5.00 |
| Hydrated Silica (Zeodent ® 124 | 4.00 |
| MCC + Guar Gum (Avicel ® CE-15) | 3.00 (0.45) |
| Surfactant - Sodium Lauryl Sulphate | 2.00 |
| Anticaries Agent - Sodium Fluoride | 0.315 |
| Sweetener - Stevia | 0.20 |
| Flavouring - Peppermint oil | 0.50 |

### Example 8—A Toothpaste Tablet Comprising Potassium Nitrate and Stannous Fluoride

| Ingredients | % w/w |
|---|---|
| Sorbitol Powder (C*PharmSorbidex ® P) | 62.846 |
| Microcrystalline Cellulose (Vivapur ® 102) | 20.00 |
| Desensitising Agent - Potassium Nitrate | 5.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate | 2.00 |
| Chelating Agent - Sodium Tripolyphosphate | 2.00 |
| Antimicrobial Agent - Stannous Fluoride | 0.454 |
| Sweetener - Stevia | 0.20 |

Stannous fluoride and potassium nitrate are difficult to formulate together in a paste. The two actives have very different requirements for stability. Stannous fluoride is highly water sensitive, while potassium nitrate is easier to formulate in a water-based composition.

The two actives can be used together in the tablets of the present invention. Both actives are stable in the composition above.

### Example 9—Gentle Whitening Formulation Including Alumina and STP

| Ingredients | % w/w |
|---|---|
| Sorbitol Powder (C*PharmSorbidex ® P) | 67.30 |
| Microcrystalline Cellulose (Vivapur ® 102) | 20.00 |
| Hydrated Silica (Zeodent ® 124) | 4.00 |
| MCC + Guar Gum (Avicel ® CE-15) | 3.00 (0.45) |
| Surfactant - Sodium Lauryl Sulphate | 2.00 |
| Pentasodium tripholyphospate (STP) | 2.00 |
| Alumina | 1.00 |
| Flavouring - Peppermint oil | 0.50 |
| Sweetener - Stevia | 0.20 |

The addition of the combination of alumina and STP to the general formulation can make an effective whitening formula. Alumina is a well-known abrasive in oral care. Pentasodium tripholyphospate (STP) is a chelating agent that helps to remove stains. It helps whiten teeth chemically, gently lifting surface stains and preventing new ones from forming.

The following examples (10 to 15) are toothpaste tablets of the invention comprising cellulose gum instead of guar gum. The surfactant in these examples is sodium methyl cocoyl taurate. The surfactant used for the guar gum examples 3 to 9 is sodium lauryl sulphate.

These combinations are not intended to be limiting, each surfactant may be used with either gum (or combination of gums) without any issue. And other surfactants may also be used.

### Example 10—A Specific Cellulose Gum Example of a Tablet of the Present Invention

| Ingredients | % w/w |
|---|---|
| Sorbitol Powder (C*PharmSorbidex ® P) | 70.30 |
| Microcrystalline Cellulose (Vivapur ® 102) | 20.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Sweetener - Stevia | 0.20 |
| Flavouring - Peppermint oil | 0.50 |

### Example 11 A Cellulose Gum Example Comprising Sodium Fluoride

| Ingredients | % w/w |
|---|---|
| Sorbitol Powder (C*PharmSorbidex ® P) | 69.985 |
| Microcrystalline Cellulose (Vivapur ® 102) | 20.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Anticaries Agent - Sodium Fluoride | 0.315 |
| Sweetener - Stevia | 0.20 |
| Flavouring - Peppermint oil | 0.50 |

### Example 12—Stannous Fluoride Example

| Ingredients | % w/w |
|---|---|
| Sorbitol Powder (C*PharmSorbidex ® P) | 67.846 |
| Microcrystalline Cellulose (Vivapur ® 102) | 20.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Chelating Agent - Sodium Tripolyphosphate | 2.00 |
| Antimicrobial Agent - Stannous Fluoride | 0.454 |
| Sweetener - Stevia | 0.20 |
| Flavouring - Peppermint oil | 0.50 |

### Example 13—A Bioglass and Fluoride in Combination

| Ingredients | % w/w |
|---|---|
| Sorbitol Powder (C*PharmSorbidex ® P) | 64.985 |
| Microcrystalline Cellulose (Vivapur ® 102) | 20.00 |
| Desensitising Agent - Novamin 4516 | 5.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Anticaries Agent - Sodium Fluoride | 0.315 |
| Sweetener - Stevia | 0.20 |
| Flavouring - Peppermint oil | 0.50 |

### Example 14—Potassium Nitrate Example

| Ingredients | % w/w |
|---|---|
| Sorbitol Powder (C*PharmSorbidex ® P) | 64.985 |
| Microcrystalline Cellulose (Vivapur ® 102) | 20.00 |
| Desensitising Agent - Potassium Nitrate | 5.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Anticaries Agent - Sodium Fluoride | 0.315 |
| Sweetener - Stevia | 0.20 |
| Flavouring - Peppermint oil | 0.50. |

### Example 15—Gentle Whitening Formulation Including Potassium Nitrate and STP

| Ingredients | % w/w |
|---|---|
| Sorbitol Powder (C*PharmSorbidex ® P) | 62.846 |
| Microcrystalline Cellulose (Vivapur ® 102) | 20.00 |
| Desensitising Agent - Potassium Nitrate | 5.00 |
| Hydrated Silica (Zeodent ® 124) | 4.00 |
| MCC + Cellulose Gum (Avicel ® PC 611) | 3.00 (0.45) |
| Surfactant - Sodium Methyl Cocoyl Taurate | 2.00 |
| Chelating Agent - Sodium Tripolyphosphate | 2.00 |
| Antimicrobial Agent - Stannous Fluoride | 0.454 |
| Sweetener - Stevia | 0.20 |
| Flavouring - Peppermint oil | 0.50 |

The formulas are embodiments of the invention and not intended to be limiting the scope of the invention. The skilled person will be aware of other alternative compositions to those above that will fall within the scope of the invention.

All of the tablets of the present invention are stable at conditions to test long term stability (40° C. and a relative humidity of 75% for 3 months) with no visible indications of degradation of the tablet appearance, nor any loss of activity of any included actives.

1. (canceled)

2. A toothpaste tablet wherein the tablet comprises;
50-80% by weight sorbitol powder;
10-30% by weight microcrystalline cellulose; and
0.1-10% by weight guar gum or cellulose gum or mixtures thereof.

3. The toothpaste tablet of claim 2 wherein the tablet further comprises at least one abrasive.

4. The toothpaste tablet of claim 3, wherein the at least one abrasive comprises between 1 and 10% by weight of the composition.

5. The toothpaste tablet of claim 2, wherein the tablet further comprises at least one surfactant.

6. The toothpaste tablet of claim 5, wherein the at least one surfactant comprises one or more surfactants from group comprising alkyl sulphates, betaines, taurates and mixtures thereof.

7. The toothpaste tablet of claim 5, wherein the at least one surfactant comprises between 1 and 10% by weight of the tablet.

8. The toothpaste tablet of claim 2, wherein the tablet comprises:
sorbitol powder between 60% and 75% by weight, microcrystalline cellulose between 15% and 25% by weight, guar gum and/or cellulose gum between 0.2% and 5% by weight, at least one abrasive between 2% and 8% by weight and at least one surfactant between 1% and 5% by weight.

9. The toothpaste tablet of claim 2, wherein the tablet comprises between 0.1% and 8% by weight of minor ingredients comprising; flavourings, whitening agents, colourants, preservatives, fragrances, pH modifiers, binders, glidants, further disintegrants and mixtures thereof.

10. The toothpaste tablet of claim 2, wherein the tablet further comprises a source of fluoride.

11. The toothpaste tablet according to claim 10, wherein the source of fluoride comprises one or more of sodium fluoride, stannous fluoride, sodium monofluorophosphate and mixtures thereof.

12-14. (canceled)

15. The toothpaste tablet of claim 2, wherein the tablet further comprises a combination of two or more potentially incompatible actives.

16. The toothpaste tablet of claim 15, wherein the combination of two or more potentially incompatible actives is selected from; a zinc salt, a potassium salt, a bioglass and stannous fluoride.

17-20. (canceled)

21. A method of making a toothpaste tablet of claim 2, comprising the steps:
a. Adding the ingredients together in a solid form
b. Mixing the ingredients together until homogeneous
c. Compacting the mixture into tablets

22. The method according to claim 21, wherein the toothpaste tablets are compacted with about 5 kN of pressure.

23. A method of cleaning teeth comprising the steps of:
a. combining the toothpaste tablet of claim 2 with a toothbrush and
b. contacting the combination of step a. with a tooth surface.
